(19) United States
(12) Patent Application Publication
(10) Pub. No.: US 2018/0009597 A1
(43) Pub. Date: Jan. 11, 2018

(54) MULTI-COMPARTMENT CONTAINER

(71) Applicant: Bradley David Sansing, Woodstock, GA (US)

(72) Inventor: Bradley David Sansing, Woodstock, GA (US)

(21) Appl. No.: 15/206,731

(22) Filed: Jul. 11, 2016

(51) Int. Cl. B65D 83/68; A61K 8/97; A61K 8/04; A61Q 17/04; B65D 83/14; B65D 83/20

(52) U.S. Cl. CPC B65D 83/68; B65D 83/752; B65D 83/20; A61K 8/046; A61Q 17/04; A61K 8/97; A61K 2800/87

(57) ABSTRACT

Disclosed herein is a multi-compartment container and methods of use thereof where the container is comprised of a first and a second aerosol chamber proximate to one another; a spray nozzle; and a spray nozzle retention device, wherein the spray nozzle is operably coupled to the spray nozzle retention device such that the spray nozzle can be operably engaged with the first aerosol chamber and then moved to separate from the first aerosol chamber and operably engage with the second aerosol chamber by movement of at least a portion of the spray nozzle retention device.

### BACKGROUND

When going to the beach or other locations where a person will be exposed to sun, a person will often carry with them several different sunscreens, sunblocks, oils, lotions, etc., including lotions for relief and/or healing if they become sunburned. This may be problematic as each sun protection factor (SPF) of sunblock has a different container, as do all the other oils, lotions, etc., making it difficult to carry each container or requiring a bag to carry numerous containers. The number of containers may be compounded if there are multiple people that will be exposed to the sun such as a family with children as each person may have special sunblock, lotions or oils that they each want to take and use.

For example, consider a family on a beach trip. A mother who wants to protect her kids from ultraviolet rays might be seen using a 50spf on the children, a father may use 30spf and a mom who wants a tan uses a 15spf. The point is a basic family does not use all of the same sunscreens. The family ends up having to carry at least three or four different sunscreen containers which are bulky, heavy and disorganized. This takes up a lot of space when packing the beach bag and suitcase.

Therefore, what is needed is a device that overcomes challenges in the art, some of which are described above. In particular, what is needed is a device that allows all of these sunscreens, sunblocks, oils, lotions, etc., to be held together and work efficiently. The device could also hold aloe vera or other lotions or medicines for sunburn relief, if it were needed.

### SUMMARY

In one aspect, a multi-compartment container is disclosed. The multi-compartment contained is comprised of a first and a second aerosol chamber proximate to one another; a spray nozzle; and a spray nozzle retention device, wherein the spray nozzle is operably coupled to the spray nozzle retention device such that the spray nozzle can be operably engaged with the first aerosol chamber and then moved to separate from the first aerosol chamber and operably engage with the second aerosol chamber by movement of at least a portion of the spray nozzle retention device.

Optionally or alternatively, the first and second aerosol chambers each contain an ultraviolet-protection substance for application to a human. For example, the ultraviolet-protection substance in the first aerosol chamber may have a sun-protection factor (SPF) that is different than the SIT of the ultraviolet-protection substance in the second aerosol chamber.

Optionally or alternatively, the first aerosol chamber contains an ultraviolet-protection substance for application to a human and the second aerosol chamber contains a sunburn treatment substance. For example, the sunburn treatment substance in the second chamber may comprise aloe vera.

Optional or alternatively, the multi-compartment container may further comprise at least a third aerosol chamber.

In one aspect, the spray nozzle retention device comprises a fixed portion and a moveable portion, wherein the moveable portion of the spray nozzle retention device is used to move the spray nozzle from the operably engaged position with the first aerosol chamber to the operably engaged position with the second aerosol chamber. Optionally or alternatively, the moveable portion is configured to be rotated such that the spray nozzle can be rotably disengaged from the operably engaged position with the first aerosol chamber rotably engaged to the operably engaged position with the second aerosol chamber. Optionally or alternatively, the moveable portion is configured to be slid such that the spray nozzle can be slidably disengaged from the operably engaged position with the first aerosol chamber and slidably engaged to the operably engaged position with the second aerosol chamber.

In one aspect, the fixed portion of the spray nozzle retention device is used to prevent movement of the first and second aerosol chambers while allowing movement of the moveable portion of the spray nozzle retention device.

Optionally or alternatively, the multi-compartment container may further comprise a container, wherein the first aerosol chamber and the second aerosol chamber are affixed within the container.

Optionally or alternatively, the multi-compartment container may further comprise a second spray nozzle, wherein the first aerosol chamber may be separated from the second aerosol chamber and the spray nozzle retention device, fitted with the second spray nozzle, and used independently from the second aerosol chamber.

In various aspects, the first and the second aerosol chamber, the spray nozzle, and the spray nozzle retention device of the multi-compartment container may be made of one or more of metal, plastic, any suitable material, or combinations thereof.

Also disclosed herein is a method of dispensing substances from a multi-compartment container comprising: providing a multi-compartment container, wherein the multi-compartment container comprises a first and a second aerosol chamber proximate to one another; a spray nozzle; and a spray nozzle retention device, wherein the spray nozzle is operably coupled to the spray nozzle retention device such that the spray nozzle can be operably engaged with the first aerosol chamber and then moved to separate from the first aerosol chamber and operably engage with the second aerosol chamber by movement of at least a portion of the spray nozzle retention device; and dispensing a first substance from the multi-compartment container when the spray nozzle is operably engaged with the first aerosol chamber and dispensing a second substance from the multi-compartment container when the spray nozzle is operably engaged with the second aerosol chamber.

Additional advantages will be set forth in part in the description which follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE DRAWINGS

The accompanying drawings, which are incorporated in and constitute a part of this specification, illustrate embodiments and together with the description, serve to explain the principles of the methods and systems:

FIG. 1 illustrates an embodiment of a disclosure of a multi-compartment container;

FIG. 2 is a cross section (A-A) of a view of the first 102 and second 104 aerosol chambers of FIG. 1;

FIG. 3 is a plan view of the multi-compartment container of FIG. 1 showing the fixed and moveable portions of the spray nozzle retention device and the spray nozzle;

FIG. 4 illustrates an embodiment of a multi-compartment container that further comprises a second spray nozzle;

FIG. 5 is another embodiment of a multi-compartment container; and

FIG. 6 is illustrates yet another embodiment of a multi-compartment container.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes [symbol] from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and systems may be understood more readily by reference to the following detailed description of preferred embodiments and the Examples included therein and to the Figures and their previous and following description.

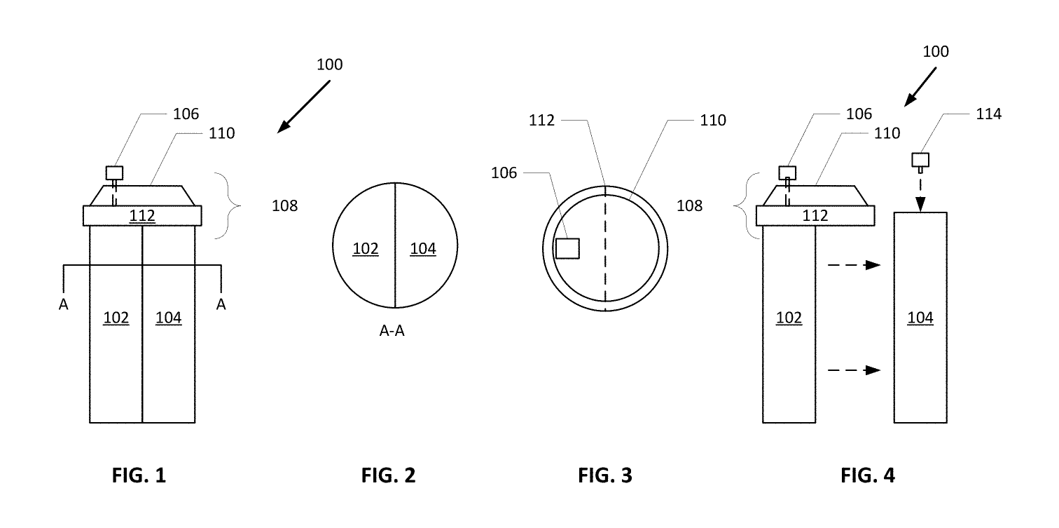

FIG. 1 illustrates an embodiment of a disclosure of a multi-compartment container 100. This embodiment is comprised of a first aerosol chamber 102 and a second aerosol chamber 104 proximate to one another. The aerosol chambers may be all or partially comprised of any suitable material such as metal, plastic, and the like. Though in FIG. 1 only two aerosol chambers 102, 104 are shown, it is to be appreciated that the multi-compartment container can be comprised of any plurality of aerosol chambers including three, four, five, etc. Further, it is to be appreciated that the aerosol chambers can have any configuration (e.g., semicircular, wedge/arc-shaped, square, rectangular, etc.) that accommodates the aerosol chambers being in close proximity to one another.

Further comprising the multi-compartment container of FIG. 1 is a single spray nozzle 106 and a spray nozzle retention device 108. The spray nozzle 106 is operably coupled to the spray nozzle retention device 108 such that the spray nozzle 106 can be operably engaged with the first aerosol chamber 102 and then separated from the first aerosol chamber 102 and operably engaged with the second aerosol chamber 104 by movement of a moveable portion 110 of the spray nozzle retention device 108.

As shown in FIG. 1, the spray nozzle retention device 108 can be comprised of a fixed portion 112 and a moveable portion 110, wherein the moveable portion 110 of the spray nozzle retention device 108 is used to move the spray nozzle 106 from the operably engaged position with the first aerosol chamber 102 to the operably engaged position with the second aerosol chamber 104. For example, the moveable portion 110 may be configured to be rotated such that the spray nozzle 106 can be rotably disengaged from the operably engaged position with the first aerosol chamber 102 and rotably engaged to the operably engaged position with the second aerosol chamber 104. In another example, the moveable portion may be configured to slide such that the spray nozzle 106 can be slidably disengaged from the operably engaged position with the first aerosol chamber 102 and slidably engaged to the operably engaged position with the second aerosol chamber 104.

As noted, the spray nozzle retention device 108 can be comprised of a moveable portion 110. In one aspect, the fixed portion 110 of the spray nozzle retention device 108 can be used to prevent movement of the first 102 and second 104 aerosol chambers while allowing movement of the moveable portion 110 of the spray nozzle retention device 108. For example, the fixed portion 112 may affix to the first 102 and second 104 aerosol chambers through compression, screw-on, tightening bands, or any other suitable means.

FIG. 2 is a cross section (A-A) of a view of the first 102 and second 104 aerosol chambers of FIG. 1 illustrating their configuration for close proximity. FIG. 3 is a plan view of the multi-compartment container of FIG. 1 showing the fixed 112 and moveable (e.g., rotable) portions of the spray nozzle retention device 108 and the spray nozzle 106.

Though not shown in any of FIGS. 1-3, in one aspect the multi-compartment container 100 can further comprise a container that encompasses or substantially encompasses the first aerosol chamber 102 and the second aerosol chamber 104. In one aspect, the first aerosol chamber 102 and the second aerosol chamber 104 are affixed within the container.

The aerosol chambers 102, 104 of FIGS. 1-3 can be used to contain various substances. For example, the first 102 and second 104 aerosol chambers each contain an ultraviolet-protection substance (i.e., sunblock, sunscreen, suntan lotion, etc.) for application to a human. In one aspect, the ultraviolet-protection substance in the first aerosol chamber 102 has a sun-protection factor (SPF) that is different than the SPF of the ultraviolet-protection substance in the second aerosol chamber 104, thus eliminating the need to carry two separate containers of ultraviolet protection substance having different SPFs. In one aspect, the first aerosol chamber 102 may contain an ultraviolet-protection substance (i.e., sunscreen or sunblock) for application to a human and the second aerosol chamber 102 may contain a sunburn treatment substance such as aloe vera.

FIG. 4 illustrates an embodiment of a multi-compartment container 100 that further comprises a second spray nozzle 114. In this embodiment, the second aerosol chamber 104 may be separated from the first aerosol chamber 102 and the spray nozzle retention device 108, fitted with the second spray nozzle 114, and used independently from the first aerosol chamber 102.

As disclosed herein, the components of the multi-compartment container 100 including the first 102 and the second 104 aerosol chamber proximate to one another, the spray nozzles 106, 114, and the spray nozzle retention device 108 can each be made of metal, plastic, any suitable material, or combinations thereof.

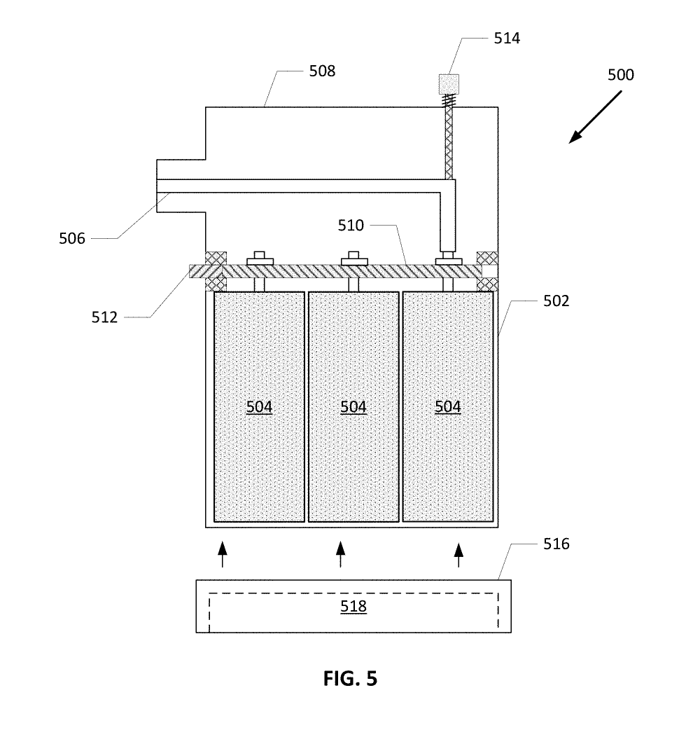

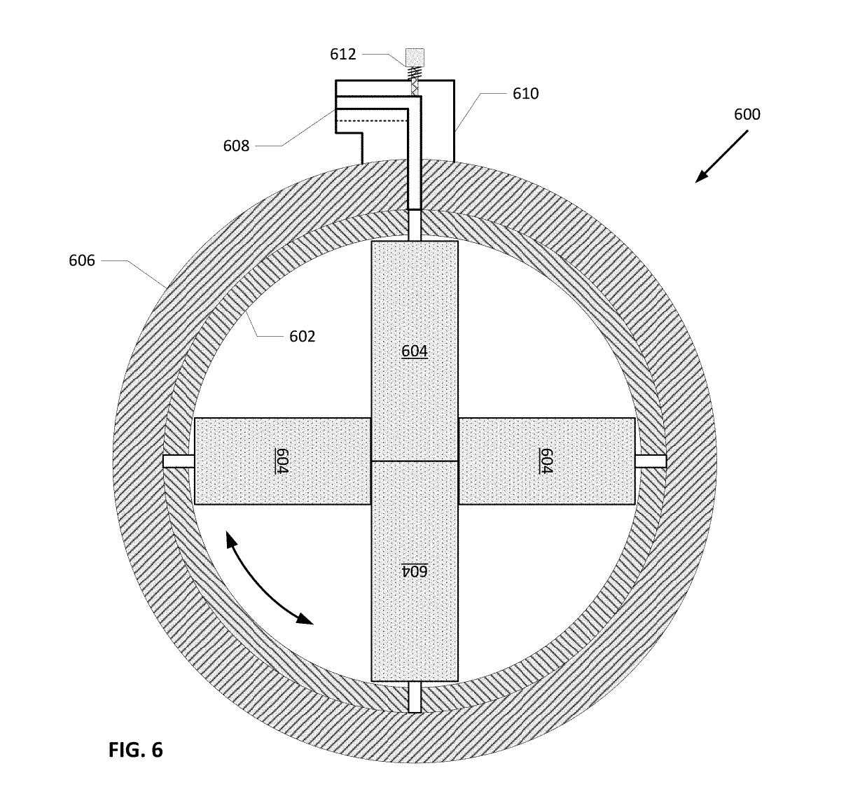

FIG. 5 illustrates another embodiment of a multi-compartment container 500. This embodiment comprises a larger container 502 that can hold a plurality of aerosol chambers 504 proximate to one another. For example, the larger container may hold two, three, four, etc. aerosol chambers 504. Further comprising the embodiment shown in FIG. 5 is a spray nozzle 506 and spray nozzle retention device 508 that includes a ring or disk 510 rotatably affixed within the larger container 502. The plurality of aerosol chambers 504 are engaged with and held in place by the ring or disk 510. The spray nozzle 506 is operably coupled to the spray nozzle retention device 508 such that the spray nozzle 506 can be operably engaged with a first aerosol chamber 504 and the ring or disk 510 can be rotated by, for example, an external mechanism 512 such as a wheel or the like, and the ring or disk 510 can be moved to separate the first aerosol chamber 504 from the spray nozzle 506 and operably engage the spray nozzle 506 with a second aerosol chamber 504 by movement of the ring or disk 510.

As shown in FIG. 5, a button 514 can be depressed, which causes release of the substance within the aerosol chamber 504 that is operably connected with the spray nozzle 506 through the spray nozzle 506. Further comprising the embodiment of FIG. 5 is a bottom 516 to the larger container 502 that further assists with holding the plurality of aerosol chambers 504 in place and provides support of the aerosol chambers 504 when button 514 is depressed. Optionally, the bottom 516 may include a compartment 518 for storing small items such as, for example, lip balm, zinc cream, keys, adhesive bandages, medicine, and the like. The bottom 516 may be removably affixed to the larger container 502 by any suitable known means including screw on/off, compression or friction fit, and the like.

In one aspect, the plurality of aerosol chambers 504 each hold a sunscreen. Each sunscreen may have different SPFs, skin sensitivity, smell, color, etc. and can be sold separately for a consumer to choose the different SPFs they need and other desirable qualities.

In one aspect, each aerosol chamber 504 can be removed from the ring or disk 510 and removed so more than one person can apply sunscreen at the same time if needed. Each aerosol chamber 504 can act individually with its own spray nozzle. Being able to remove an individual sunscreen container also allows the consumer to purchase the sunscreen they run out of quicker and replace when needed.

In one aspect, the larger container 502 may also have a handle attached to it so it will be easier for the consumer to hold the device. This handle will allow the consumer to spray the sunscreen needed easier and more efficiently over the body. This will allow them to spray the sunscreen in hard to reach areas like the back. The button that is pushed to spray the sunscreen needed is located on top of this handle where only one hand is needed to disperse the sunscreen.

FIG. 6 illustrates yet another embodiment of a multi-compartment container 600. This embodiment comprises a first ring device 602 that holds together a plurality of aerosol sunscreen containers 604 with different sunscreens. Each sunscreen container 604 locks into a specific space within the first ring device 602. The first ring device 604 is rotatably affixed within a second ring 606. Further comprising the second ring 606 is a spray nozzle 608 and a spray nozzle retention device 610. The plurality of aerosol chambers 604 are engaged with and held in place by the first ring device 602. The spray nozzle 608 is operably coupled to the spray nozzle retention device 610 such that the spray nozzle 608 can be operably engaged with a first aerosol chamber 604 and the first ring device 602 can be rotated to separate the first aerosol chamber 604 from the spray nozzle 608 and operably engage the spray nozzle 608 with a second aerosol chamber 604 by movement of the first ring device 602.

As shown in FIG. 6, a button 612 can be depressed, which causes release of the substance within the aerosol chamber 604 that is operably connected with the spray nozzle 608. In one aspect, the plurality of aerosol chambers 604 each hold a sunscreen. Each sunscreen may have different SPFs, skin sensitivity, smell, color, etc. and can be sold separately for a consumer to choose the different SPFs they need and other desirable qualities.

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order.

Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made without departing from the scope or spirit. Other embodiments will be apparent to those skilled in the art from consideration of the specification and practice disclosed herein. It is intended that the specification and examples he considered as exemplary only, with a true scope and spirit being indicated by the following claims.

What is claimed is:

1. A multi-compartment container comprised of:
a first and a second aerosol chamber proximate to one another;
a spray nozzle; and
a spray nozzle retention device, wherein the spray nozzle is operably coupled to the spray nozzle retention device such that the spray nozzle can be operably engaged with the first aerosol chamber and then moved to separate from the first aerosol chamber and operably engage with the second aerosol chamber by movement of at least a portion of the spray nozzle retention device.

2. The multi-compartment container of claim 1, wherein the first and second aerosol chambers each contain an ultraviolet-protection substance for application to a human.

3. The multi-compartment container of claim 2, wherein the ultraviolet-protection substance in the first aerosol chamber has a sun-protection factor (SPF) that is different than the SET of the ultraviolet-protection substance in the second aerosol chamber.

4. The multi-compartment container of claim 1, wherein the first aerosol chamber contains an ultraviolet-protection substance for application to a human and the second aerosol chamber contains a sunburn treatment substance.

5. The multi-compartment container of claim 4, wherein the sunburn treatment substance comprises aloe vera.

6. The multi-compartment container of claim 1, further comprising at least a third aerosol chamber.

7. The multi-compartment container of claim 1, wherein the spray nozzle retention device comprises a fixed portion and a moveable portion, wherein the moveable portion of the spray nozzle retention device is used to move the spray nozzle from the operably engaged position with the first aerosol chamber to the operably engaged position with the second aerosol chamber.

8. The multi-compartment container of claim 7, wherein the moveable portion is configured to be rotated such that the spray nozzle can be rotably disengaged from the operably engaged position with the first aerosol chamber rotably engaged to the operably engaged position with the second aerosol chamber.

9. The W.-compartment container of claim 7, wherein the moveable portion is configured to be slid such that the spray nozzle can be slidably disengaged from the operably engaged position with the first aerosol chamber and slidably engaged to the operably engaged position with the second aerosol chamber.

10. The multi-compartment container of claim 1, wherein the fixed portion of the spray nozzle retention device is used to prevent movement of the first and second aerosol chambers while allowing movement of the moveable portion of the spray nozzle retention device.

11. The multi-compartment container of claim 1, further comprising a container, wherein the first aerosol chamber and the second aerosol chamber are affixed within the container.

12. The multi-compartment container of claim 1, further comprising a second spray nozzle, wherein the first aerosol clamber may be separated from the second aerosol chamber and the spray nozzle retention device, fitted with the second spray nozzle, and used independently from the second aerosol chamber.

13. The multi-compartment container of claim 1, wherein the first and the second aerosol chamber, the spray nozzle, and the spray nozzle retention device are each made of one or more of metal, plastic, any suitable material, or combinations thereof.

14. A method of dispensing substances from a multi-compartment container comprising:
providing a multi-compartment container, wherein the multi-compartment container comprises a first and a second aerosol chamber proximate to one another; a spray nozzle; and a spray nozzle retention device, wherein the spray nozzle is operably coupled to the spray nozzle retention device such that the spray nozzle can be operably engaged with the first aerosol chamber and then moved to separate from the first aerosol chamber and operably engage with the second aerosol chamber by movement of at least a portion of the spray nozzle retention device; and
dispensing a first substance from the multi-compartment container when the spray nozzle is operably engaged with the first aerosol chamber and dispensing a second substance from the multi-compartment container when the spray nozzle is operably engaged with the second aerosol chamber,

15. The method of claim 14, wherein the first and second aerosol chambers each contain an ultraviolet-protection substance for application to a human.

16. The method of claim 15, wherein the ultraviolet-protection substance in the first aerosol chamber has a sun-protection factor (SPF) that is different than the SPF of the ultraviolet-protection substance in the second aerosol chamber.

17. The method of claim 14, wherein the first aerosol chamber contains an ultraviolet-protection substance for application to a human and the second aerosol chamber contains a sunburn treatment substance.

18. The method of claim 17, wherein the sunburn treatment substance comprises aloe vera.

19. The method of claim 14, wherein the multi-compartment container further comprises at least a third aerosol chamber.

20. The method of claim 14, wherein the spray nozzle retention device comprises a fixed portion and a moveable portion, wherein the moveable portion of the spray nozzle retention device is used to move the spray nozzle from the operably engaged position with the first aerosol chamber to the operably engaged position with the second aerosol chamber.

21. The method of claim 20, wherein the moveable portion is configured to be rotated such that the spray nozzle can be rotably disengaged from the operably engaged position with the first aerosol chamber rotably engaged to the operably engaged position with the second aerosol chamber.

22. The method of claim 20, wherein the moveable portion is configured to be slid such that the spray nozzle can be slidably disengaged from the operably engaged position with the first aerosol chamber and slidably engaged to the operably engaged position with the second aerosol chamber.

23. The method of claim 20, wherein the fixed portion of the spray nozzle retention device is used to prevent movement of the first and second aerosol chambers while allowing movement of the moveable portion of the spray nozzle retention device.

24. The method of claim 14, wherein the multi-compartment container further comprises a container, wherein the first aerosol chamber and the second aerosol chamber are affixed within the container.

25. The method of claim 14, wherein the multi-compartment container further comprises a second spray nozzle, wherein the first aerosol chamber may be separated from the second aerosol chamber and the spray nozzle retention device, fitted with the second spray nozzle, and used independently from the second aerosol chamber.

26. The method of claim 14, wherein the first and the second aerosol chamber proximate to one another, the spray nozzle, and the spray nozzle retention device are each made of metal, plastic, any suitable material, or combinations thereof.
